SEQUENCE LISTING (150 sequences)

SEQ ID NO: 1 - DNA, 33 bp, Artificial Sequence
  Synthetic polynucleotide
tcggtcagaggctagctacaacgatgcgttgct

SEQ ID NO: 2 - DNA, 33 bp, Artificial Sequence
  Synthetic polynucleotide
ggcgtacgaggctagctacaacgactgctcggt

SEQ ID NO: 3 - DNA, 33 bp, Artificial Sequence
  Synthetic polynucleotide
ggcggcgtaggctagctacaacgagacctgctc

SEQ ID NO: 4 - DNA, 33 bp, Artificial Sequence
  Synthetic polynucleotide
ctcgggtcaggctagctacaacgactgggtagc

SEQ ID NO: 5 - DNA, 33 bp, Artificial Sequence
  Synthetic polynucleotide
tcctctgcaggctagctacaacgacggggtcct

SEQ ID NO: 6 - DNA, 33 bp, Artificial Sequence
  Synthetic polynucleotide
actctgcaaggctagctacaacgatctgcgagc

SEQ ID NO: 7 - DNA, 33 bp, Artificial Sequence
  Synthetic polynucleotide
gggcgacgaggctagctacaacgatctgcaatt

SEQ ID NO: 8 - DNA, 33 bp, Artificial Sequence
  Synthetic polynucleotide
aaggggcgaggctagctacaacgagactctgca

SEQ ID NO: 9 - DNA, 33 bp, Artificial Sequence
  Synthetic polynucleotide
aaaacgggaggctagctacaacgacaggttgta

SEQ ID NO: 10 - DNA, 33 bp, Artificial Sequence
  Synthetic polynucleotide
agaataaaaggctagctacaacgagggaccagg

SEQ ID NO: 11 - DNA, 33 bp, Artificial Sequence
  Synthetic polynucleotide
atggcagaaggctagctacaacgaaaaacggga

SEQ ID NO: 12 - DNA, 33 bp, Artificial Sequence
  Synthetic polynucleotide
aactgggtaggctagctacaacgaggcagaata

SEQ ID NO: 13 - DNA, 33 bp, Artificial Sequence
  Synthetic polynucleotide
atccaaaaaggctagctacaacgatgggtatgg

SEQ ID NO: 14 - DNA, 33 bp, Artificial Sequence
  Synthetic polynucleotide
aggggaagaggctagctacaacgaaaaaatcca

SEQ ID NO: 15 - DNA, 33 bp, Artificial Sequence
  Synthetic polynucleotide
ttttaaaaaggctagctacaacgatatcttgga

SEQ ID NO: 16 - DNA, 33 bp, Artificial Sequence
  Synthetic polynucleotide
gtggggggaggctagctacaacgagggaaggct

SEQ ID NO: 17 - DNA, 33 bp, Artificial Sequence
  Synthetic polynucleotide
gttgaatgaggctagctacaacgattgctttcg

SEQ ID NO: 18 - DNA, 33 bp, Artificial Sequence
  Synthetic polynucleotide
gtcgttgaaggctagctacaacgagatttgctt

SEQ ID NO: 19 - DNA, 33 bp, Artificial Sequence
  Synthetic polynucleotide
ggcccggaaggctagctacaacgaccgcgcgcg

SEQ ID NO: 20 - DNA, 33 bp, Artificial Sequence
  Synthetic polynucleotide
tcacctccaggctagctacaacgaggcctcggc

SEQ ID NO: 21 - DNA, 33 bp, Artificial Sequence
  Synthetic polynucleotide
ccgccgtcaggctagctacaacgactccatggc

SEQ ID NO: 22 - DNA, 33 bp, Artificial Sequence
  Synthetic polynucleotide
ggtggctcaggctagctacaacgaccagcgcgg

SEQ ID NO: 23 - DNA, 33 bp, Artificial Sequence
  Synthetic polynucleotide
cgttgagcaggctagctacaacgaggcggggtg

SEQ ID NO: 24 - DNA, 33 bp, Artificial Sequence
  Synthetic polynucleotide
ccgcgtccaggctagctacaacgagtaggagtg

SEQ ID NO: 25 - DNA, 33 bp, Artificial Sequence
  Synthetic polynucleotide
cagcgggtaggctagctacaacgatgcgccgcg

SEQ ID NO: 26 - DNA, 33 bp, Artificial Sequence
  Synthetic polynucleotide
gcacatccaggctagctacaacgactcctccgg

SEQ ID NO: 27 - DNA, 33 bp, Artificial Sequence
  Synthetic polynucleotide
aaaagcacaggctagctacaacgaccacctcct

SEQ ID NO: 28 - DNA, 33 bp, Artificial Sequence
  Synthetic polynucleotide
taaaaagcaggctagctacaacgaatccacctc

SEQ ID NO: 29 - DNA, 33 bp, Artificial Sequence
  Synthetic polynucleotide
gaccgtcgaggctagctacaacgagttaaaaag

SEQ ID NO: 30 - DNA, 33 bp, Artificial Sequence
  Synthetic polynucleotide
ttgccttgaggctagctacaacgacgtcgatgt

SEQ ID NO: 31 - DNA, 33 bp, Artificial Sequence
  Synthetic polynucleotide
agggcgggaggctagctacaacgagtggttgcc

SEQ ID NO: 32 - DNA, 33 bp, Artificial Sequence
  Synthetic polynucleotide
tggccctgaggctagctacaacgacgagtttcc

SEQ ID NO: 33 - DNA, 33 bp, Artificial Sequence
  Synthetic polynucleotide
acctctgcaggctagctacaacgacgtggccct

SEQ ID NO: 34 - DNA, 33 bp, Artificial Sequence
  Synthetic polynucleotide
cggagggtaggctagctacaacgactctgcacc

SEQ ID NO: 35 - DNA, 33 bp, Artificial Sequence
  Synthetic polynucleotide
ggcggcacaggctagctacaacgactggctccc

SEQ ID NO: 36 - DNA, 33 bp, Artificial Sequence
  Synthetic polynucleotide
cgggcggcaggctagctacaacgaacctggctc

SEQ ID NO: 37 - DNA, 33 bp, Artificial Sequence
  Synthetic polynucleotide
agggatccaggctagctacaacgagaagcagag

SEQ ID NO: 38 - DNA, 33 bp, Artificial Sequence
  39
gggtagggaggctagctacaacgaccatgaagc

SEQ ID NO: 39 - DNA, 33 bp, Artificial Sequence
  Synthetic polynucleotide
gggctgagaggctagctacaacgatccaggggg

SEQ ID NO: 40 - DNA, 33 bp, Artificial Sequence
  Synthetic polynucleotide
gtggatggaggctagctacaacgagtcttggag

SEQ ID NO: 41 - DNA, 33 bp, Artificial Sequence
  42
cgtggtggaggctagctacaacgaggacgtctt

SEQ ID NO: 42 - DNA, 33 bp, Artificial Sequence
  Synthetic polynucleotide
gggggtagaggctagctacaacgaggagagggg

SEQ ID NO: 43 - DNA, 33 bp, Artificial Sequence
  Synthetic polynucleotide
ggaggaggaggctagctacaacgagaggccggg

SEQ ID NO: 44 - DNA, 33 bp, Artificial Sequence
  Synthetic polynucleotide
gccccccgaggctagctacaacgaaaggaggag

SEQ ID NO: 45 - DNA, 33 bp, Artificial Sequence
  Synthetic polynucleotide
ccggggagaggctagctacaacgagtccttcgg

SEQ ID NO: 46 - DNA, 33 bp, Artificial Sequence
  Synthetic polynucleotide
ggacagcgaggctagctacaacgagggtccggg

SEQ ID NO: 47 - DNA, 33 bp, Artificial Sequence
  Synthetic polynucleotide
tggggtggaggctagctacaacgaagcgatggg

SEQ ID NO: 48 - DNA, 33 bp, Artificial Sequence
  Synthetic polynucleotide
cttgaggcaggctagctacaacgatctttctcg

SEQ ID NO: 49 - DNA, 33 bp, Artificial Sequence
  Synthetic polynucleotide
cacctggtaggctagctacaacgattgaggcac

SEQ ID NO: 50 - DNA, 33 bp, Artificial Sequence
  Synthetic polynucleotide
gcaggggcaggctagctacaacgactggtactt

SEQ ID NO: 51 - DNA, 33 bp, Artificial Sequence
  Synthetic polynucleotide
ccagcttcaggctagctacaacgagctgtcggg

SEQ ID NO: 52 - DNA, 33 bp, Artificial Sequence
  Synthetic polynucleotide
gtgggacgaggctagctacaacgatccagcttc

SEQ ID NO: 53 - DNA, 33 bp, Artificial Sequence
  Synthetic polynucleotide
ggagtgggaggctagctacaacgagactccagc

SEQ ID NO: 54 - DNA, 33 bp, Artificial Sequence
  Synthetic polynucleotide
atgctgccaggctagctacaacgagggagtggg

SEQ ID NO: 55 - DNA, 33 bp, Artificial Sequence
  Synthetic polynucleotide
gggcggtcaggctagctacaacgagctgccacg

SEQ ID NO: 56 - DNA, 33 bp, Artificial Sequence
  Synthetic polynucleotide
gaggctccaggctagctacaacgaccagggcgg

SEQ ID NO: 57 - DNA, 33 bp, Artificial Sequence
  Synthetic polynucleotide
gtgggtcgaggctagctacaacgagaggaggct

SEQ ID NO: 58 - DNA, 33 bp, Artificial Sequence
  Synthetic polynucleotide
aggtggtgaggctagctacaacgaggggtggtg

SEQ ID NO: 59 - DNA, 33 bp, Artificial Sequence
  Synthetic polynucleotide
actcgggcaggctagctacaacgagtagggcgg

SEQ ID NO: 60 - DNA, 33 bp, Artificial Sequence
  Synthetic polynucleotide
ggagctgtaggctagctacaacgatcgggcacg

SEQ ID NO: 61 - DNA, 33 bp, Artificial Sequence
  Synthetic polynucleotide
ggacttgcaggctagctacaacgaccgaagccg

SEQ ID NO: 62 - DNA, 33 bp, Artificial Sequence
  Synthetic polynucleotide
gggcctggaggctagctacaacgattgcatccg

SEQ ID NO: 63 - DNA, 33 bp, Artificial Sequence
  Synthetic polynucleotide
tgtgctggaggctagctacaacgacgggccttg

SEQ ID NO: 64 - DNA, 33 bp, Artificial Sequence
  Synthetic polynucleotide
gttcacacaggctagctacaacgatccctgcct

SEQ ID NO: 65 - DNA, 33 bp, Artificial Sequence
  Synthetic polynucleotide
cagttcacaggctagctacaacgaactccctgc

SEQ ID NO: 66 - DNA, 33 bp, Artificial Sequence
  Synthetic polynucleotide
cacagttcaggctagctacaacgaacactccct

SEQ ID NO: 67 - DNA, 33 bp, Artificial Sequence
  Synthetic polynucleotide
gttgccccaggctagctacaacgaagttcacac

SEQ ID NO: 68 - DNA, 33 bp, Artificial Sequence
  Synthetic polynucleotide
tcgccgccaggctagctacaacgaagtggggtc

SEQ ID NO: 69 - DNA, 33 bp, Artificial Sequence
  Synthetic polynucleotide
cccgtgccaggctagctacaacgactcgccgcc

SEQ ID NO: 70 - DNA, 33 bp, Artificial Sequence
  71
ggcgttgcaggctagctacaacgaaggtagtgt

SEQ ID NO: 71 - DNA, 33 bp, Artificial Sequence
  Synthetic polynucleotide
tggcttctaggctagctacaacgagccctcgtc

SEQ ID NO: 72 - DNA, 33 bp, Artificial Sequence
  Synthetic polynucleotide
gggctctgaggctagctacaacgagcctggctt

SEQ ID NO: 73 - DNA, 33 bp, Artificial Sequence
  Synthetic polynucleotide
gggaccccaggctagctacaacgacggagcccg

SEQ ID NO: 74 - DNA, 33 bp, Artificial Sequence
  Synthetic polynucleotide
ggtgggggaggctagctacaacgacccaccgga

SEQ ID NO: 75 - DNA, 33 bp, Artificial Sequence
  Synthetic polynucleotide
ggcgggggaggctagctacaacgaccgagggcc

SEQ ID NO: 76 - DNA, 33 bp, Artificial Sequence
  Synthetic polynucleotide
gggctgggaggctagctacaacgagggcaggga

SEQ ID NO: 77 - DNA, 33 bp, Artificial Sequence
  Synthetic polynucleotide
cgtcgaggaggctagctacaacgaccgcccctc

SEQ ID NO: 78 - DNA, 33 bp, Artificial Sequence
  Synthetic polynucleotide
gggctggcaggctagctacaacgacttcccgta

SEQ ID NO: 79 - DNA, 33 bp, Artificial Sequence
  Synthetic polynucleotide
cgatgcccaggctagctacaacgaccggggcgg

SEQ ID NO: 80 - DNA, 33 bp, Artificial Sequence
  Synthetic polynucleotide
gctccacgaggctagctacaacgagcccatccg

SEQ ID NO: 81 - DNA, 33 bp, Artificial Sequence
  Synthetic polynucleotide
ccggctccaggctagctacaacgagatgcccat

SEQ ID NO: 82 - DNA, 33 bp, Artificial Sequence
  Synthetic polynucleotide
tctccgcaaggctagctacaacgaccggctcca

SEQ ID NO: 83 - DNA, 33 bp, Artificial Sequence
  Synthetic polynucleotide
ccgtcagcaggctagctacaacgagtctccgca

SEQ ID NO: 84 - DNA, 33 bp, Artificial Sequence
  Synthetic polynucleotide
tccccggcaggctagctacaacgacggctcggt

SEQ ID NO: 85 - DNA, 33 bp, Artificial Sequence
  Synthetic polynucleotide
cccccgcgaggctagctacaacgagctcgtccg

SEQ ID NO: 86 - DNA, 33 bp, Artificial Sequence
  Synthetic polynucleotide
gtagggagaggctagctacaacgacccaggctg

SEQ ID NO: 87 - DNA, 33 bp, Artificial Sequence
  Synthetic polynucleotide
gggcgggcaggctagctacaacgacaaggcgcc

SEQ ID NO: 88 - DNA, 33 bp, Artificial Sequence
  Synthetic polynucleotide
cgggaaggaggctagctacaacgatcgcccgcg

SEQ ID NO: 89 - DNA, 33 bp, Artificial Sequence
  Synthetic polynucleotide
tagtcctcaggctagctacaacgagcggccccg

SEQ ID NO: 90 - DNA, 33 bp, Artificial Sequence
  Synthetic polynucleotide
tccccgacaggctagctacaacgactccagtcc

SEQ ID NO: 91 - DNA, 33 bp, Artificial Sequence
  Synthetic polynucleotide
tttccccgaggctagctacaacgaacctccagt

SEQ ID NO: 92 - DNA, 33 bp, Artificial Sequence
  Synthetic polynucleotide
tgagcgcgaggctagctacaacgacctcagttt

SEQ ID NO: 93 - DNA, 33 bp, Artificial Sequence
  Synthetic polynucleotide
ggaccacaaggctagctacaacgaaggtggttg

SEQ ID NO: 94 - DNA, 33 bp, Artificial Sequence
  Synthetic polynucleotide
cttggaccaggctagctacaacgaaacaggtgg

SEQ ID NO: 95 - DNA, 33 bp, Artificial Sequence
  Synthetic polynucleotide
aaacttggaggctagctacaacgacacaacagg

SEQ ID NO: 96 - DNA, 33 bp, Artificial Sequence
  Synthetic polynucleotide
ctgattaaaggctagctacaacgattggaccac

SEQ ID NO: 97 - DNA, 33 bp, Artificial Sequence
  Synthetic polynucleotide
tggtgctgaggctagctacaacgataaacttgg

SEQ ID NO: 98 - DNA, 33 bp, Artificial Sequence
  Synthetic polynucleotide
tgatgatcaggctagctacaacgactctgtctg

SEQ ID NO: 99 - DNA, 33 bp, Artificial Sequence
  Synthetic polynucleotide
tggtgatgaggctagctacaacgacatctctgt

SEQ ID NO: 100 - DNA, 33 bp, Artificial Sequence
  Synthetic polynucleotide
gcttggtgaggctagctacaacgagatcatctc

SEQ ID NO: 101 - DNA, 33 bp, Artificial Sequence
  Synthetic polynucleotide
atgggaacaggctagctacaacgaccgccgtcc

SEQ ID NO: 102 - DNA, 33 bp, Artificial Sequence
  Synthetic polynucleotide
gaatgggaaggctagctacaacgaatccgccgt

SEQ ID NO: 103 - DNA, 33 bp, Artificial Sequence
  Synthetic polynucleotide
tgacaggaaggctagctacaacgagggaacatc

SEQ ID NO: 104 - DNA, 33 bp, Artificial Sequence
  Synthetic polynucleotide
agtaaatgaggctagctacaacgaaggaatggg

SEQ ID NO: 105 - DNA, 33 bp, Artificial Sequence
  Synthetic polynucleotide
cacagtaaaggctagctacaacgagacaggaat

SEQ ID NO: 106 - DNA, 33 bp, Artificial Sequence
  Synthetic polynucleotide
gcccggccaggctagctacaacgaagtaaatga

SEQ ID NO: 107 - DNA, 33 bp, Artificial Sequence
  Synthetic polynucleotide
ccacaaacaggctagctacaacgacctgtagtg

SEQ ID NO: 108 - DNA, 33 bp, Artificial Sequence
  Synthetic polynucleotide
gtccacaaaggctagctacaacgaatcctgtag

SEQ ID NO: 109 - DNA, 33 bp, Artificial Sequence
  Synthetic polynucleotide
ccacgtccaggctagctacaacgaaaacatcct

SEQ ID NO: 110 - DNA, 33 bp, Artificial Sequence
  Synthetic polynucleotide
ccaagaccaggctagctacaacgagtccacaaa

SEQ ID NO: 111 - DNA, 33 bp, Artificial Sequence
  Synthetic polynucleotide
ccaccaagaggctagctacaacgacacgtccac

SEQ ID NO: 112 - DNA, 33 bp, Artificial Sequence
  Synthetic polynucleotide
gctggtccaggctagctacaacgacaagaccac

SEQ ID NO: 113 - DNA, 33 bp, Artificial Sequence
  Synthetic polynucleotide
gctctggtaggctagctacaacgacgccagtgg

SEQ ID NO: 114 - DNA, 33 bp, Artificial Sequence
  Synthetic polynucleotide
ctgcacccaggctagctacaacgattgccgctc

SEQ ID NO: 115 - DNA, 33 bp, Artificial Sequence
  Synthetic polynucleotide
cacactgcaggctagctacaacgaccacttgcc

SEQ ID NO: 116 - DNA, 33 bp, Artificial Sequence
  Synthetic polynucleotide
ctttccacaggctagctacaacgatgcacccac

SEQ ID NO: 117 - DNA, 33 bp, Artificial Sequence
  Synthetic polynucleotide
gcctttccaggctagctacaacgaactgcaccc

SEQ ID NO: 118 - DNA, 33 bp, Artificial Sequence
  Synthetic polynucleotide
ttcctggcaggctagctacaacgagctgccctc

SEQ ID NO: 119 - DNA, 33 bp, Artificial Sequence
  Synthetic polynucleotide
gtggacgtaggctagctacaacgaaggcggttt

SEQ ID NO: 120 - DNA, 33 bp, Artificial Sequence
  Synthetic polynucleotide
ccgggtggaggctagctacaacgagtacaggcg

SEQ ID NO: 121 - DNA, 33 bp, Artificial Sequence
  Synthetic polynucleotide
cctggcgcaggctagctacaacgaccagtgcgc

SEQ ID NO: 122 - DNA, 33 bp, Artificial Sequence
  Synthetic polynucleotide
caaatgaaaggctagctacaacgattcctggcg

SEQ ID NO: 123 - DNA, 33 bp, Artificial Sequence
  Synthetic polynucleotide
tttcccaaaggctagctacaacgagaaacttcc

SEQ ID NO: 124 - DNA, 33 bp, Artificial Sequence
  Synthetic polynucleotide
attgttggaggctagctacaacgagcccccttg

SEQ ID NO: 125 - DNA, 33 bp, Artificial Sequence
  Synthetic polynucleotide
tgggtcacaggctagctacaacgatgttggacg

SEQ ID NO: 126 - DNA, 33 bp, Artificial Sequence
  Synthetic polynucleotide
tctgggtcaggctagctacaacgaattgttgga

SEQ ID NO: 127 - DNA, 33 bp, Artificial Sequence
  Synthetic polynucleotide
gcacaatcaggctagctacaacgactgggtcac

SEQ ID NO: 128 - DNA, 33 bp, Artificial Sequence
  Synthetic polynucleotide
ggagcacaaggctagctacaacgacatctgggt

SEQ ID NO: 129 - DNA, 33 bp, Artificial Sequence
  Synthetic polynucleotide
actggagcaggctagctacaacgaaatcatctg

SEQ ID NO: 130 - DNA, 33 bp, Artificial Sequence
  Synthetic polynucleotide
atggagggaggctagctacaacgatggagcaca

SEQ ID NO: 131 - DNA, 33 bp, Artificial Sequence
  Synthetic polynucleotide
tggtacttaggctagctacaacgaggagggact

SEQ ID NO: 132 - DNA, 33 bp, Artificial Sequence
  Synthetic polynucleotide
gggctggtaggctagctacaacgattatggagg

SEQ ID NO: 133 - DNA, 33 bp, Artificial Sequence
  Synthetic polynucleotide
tcaacgataggctagctacaacgagcagccggg

SEQ ID NO: 134 - DNA, 33 bp, Artificial Sequence
  Synthetic polynucleotide
cctcaacgaggctagctacaacgaatgcagccg

SEQ ID NO: 135 - DNA, 33 bp, Artificial Sequence
  Synthetic polynucleotide
tcacctcaaggctagctacaacgagatatgcag

SEQ ID NO: 136 - DNA, 33 bp, Artificial Sequence
  Synthetic polynucleotide
cgtcgttcaggctagctacaacgactcaacgat

SEQ ID NO: 137 - DNA, 33 bp, Artificial Sequence
  Synthetic polynucleotide
gtaaagataggctagctacaacgagcgtgttgg

SEQ ID NO: 138 - DNA, 33 bp, Artificial Sequence
  Synthetic polynucleotide
aagtaaagaggctagctacaacgaatgcgtgtt

SEQ ID NO: 139 - DNA, 33 bp, Artificial Sequence
  Synthetic polynucleotide
ggcaatgaaggctagctacaacgatgggtttct

SEQ ID NO: 140 - DNA, 33 bp, Artificial Sequence
  Synthetic polynucleotide
tcacggcaaggctagctacaacgagaactgggt

SEQ ID NO: 141 - DNA, 33 bp, Artificial Sequence
  Synthetic polynucleotide
aggcagtcaggctagctacaacgaggcaatgaa

SEQ ID NO: 142 - DNA, 33 bp, Artificial Sequence
  Synthetic polynucleotide
atctcggcaggctagctacaacgatctggtagg

SEQ ID NO: 143 - DNA, 33 bp, Artificial Sequence
  Synthetic polynucleotide
gctgagtaaggctagctacaacgactcggcatt

SEQ ID NO: 144 - DNA, 33 bp, Artificial Sequence
  Synthetic polynucleotide
tattatcaaggctagctacaacgatttcagctg

SEQ ID NO: 145 - DNA, 33 bp, Artificial Sequence
  Synthetic polynucleotide
gggttattaggctagctacaacgacaattttca

SEQ ID NO: 146 - DNA, 33 bp, Artificial Sequence
  Synthetic polynucleotide
aaggggttaggctagctacaacgatatcaattt

SEQ ID NO: 147 - DNA, 33 bp, Artificial Sequence
  Synthetic polynucleotide
ctcccggaaggctagctacaacgacctttggca

SEQ ID NO: 148 - DNA, 33 bp, Artificial Sequence
  Synthetic polynucleotide
gtacatggaggctagctacaacgatcaaagttc

SEQ ID NO: 149 - DNA, 15 bp, Artificial Sequence
  Synthetic polynucleotide
ggctagctacaacga

SEQ ID NO: 150 - DNA, 33 bp, Artificial Sequence
  Synthetic polynucleotide
nnnnnnnnnggctagctacaacgannnnnnnnn

(19) United States
(12) Patent Application Publication
(10) Pub. No.: US 2014/0248377 A1
(43) Pub. Date: Sep. 4, 2014

(54) MULTIPLE EMULSION

(71) Applicants: Frank Runkel, Buseck (DE); Thomas Schmidts, Giessen (DE)

(72) Inventors: Frank Runkel, Buseck (DE); Thomas Schmidts, Giessen (DE)

(73) Assignee: STERNA BIOLOGICALS GMBH & CO. KG BIOMEDIZINISCHES FORSCHUNGSZENTRUM, Marburg (DE)

(21) Appl. No.: 14/237,809

(22) PCT Filed: Aug. 2, 2012
(86) PCT No.: PCT/EP2012/065200

§ 371 Date: May 14, 2014

(30) Foreign Application Priority Data
Aug. 10, 2011 (DE) 10 2011 109 868.6

(51) Int. Cl. A61K 9/113; A61K 8/06; A61K 8/60; A61K 8/23; A61K 31/713; A61K 33/06

(52) U.S. Cl. CPC A61K 9/113; A61K 31/713; A61K 33/06; A61K 8/606; A61K 8/23; A61K 8/066
USPC 424/709; 514/44 R

(57) ABSTRACT

The invention relates to a multiple emulsion for the application of at least one pharmaceutical or cosmetic active ingredient which has an external water phase W₁, an oil phase O dispersed in the external water phase W₁ and an internal water phase W₂ dispersed in the oil phase O, where, in the internal water phase W₂, at least one electrolyte selected from the group of the alkali metal and alkaline earth metal halides and sulphates and at least one hydrophilic active ingredient is provided. In order to achieve a high stability of the emulsion, the invention envisages that the external water phase comprises a hydrophilic emulsifier which is a polymer of ethylene oxide and propylene oxide, and that the oil phase O is formed by triacylglycerides and has a lipophilic emulsifier from the group of dimethicones. Also provided is the use of such a multiple emulsion for producing a medicament, a medicinal product or a cosmetic, in particular a medicament for topical use or for application of the active ingredient via mucosa or for the intraperitoneal application of the active ingredient.

The invention relates to a multiple emulsion according to the preamble of claim 1, to the use of a multiple emulsion for producing a pharmaceutical, a medicinal product or a cosmetic according to claim 17, and to a pharmaceutical comprising a multiple emulsion according to claim 19 or 21.

Multiple water-in-oil-in-water emulsions (W/O/W emulsions) are complex systems, which comprise an external water phase and a water-in-oil emulsion dispersed therein, so that in total both a water-in-oil emulsion and an oil-in-water emulsion are present.

As a result of the properties associated therewith, multiple emulsions are suitable for the application of pharmaceutical or cosmetic active ingredients, wherein in particular sensitive active ingredients may be entrapped in the internal aqueous phase of a multiple emulsion. In this way, the active ingredients can not only be protected from outside influences and degradation, but also be released with delay.

Various multiple emulsions are already described in the prior art. However, one fundamental problem of such multiple emulsions is that these are frequently unstable due to the interfaces present at the phase boundaries between the water phase and the oil phase. As a result, drastically changed viscosity, separation or inversion of the phases, or loss of the multiplicity may occur over time.

The stability of a multiple emulsion is influenced by multiple parameters. Schmidts et al. (T. Schmidts, D. Dobler, P. Schlupp, C. Nissing, H. Garn, F. Runkel, International Journal of Pharmaceutics 398 (2010) 107-113), for example, described the influence of the composition of an internal water phase that is dispersed in the oil phase on the stability of a multiple emulsion. It was found that the selection of the emulsifier has particularly great influence on the stability, but also the nature of the electrolyte showed a clear effect. When a composition of a multiple emulsion is changed, it is thus usually not possible to assess whether, and if so, in what way, the properties such as stability, viscosity, drop size and the like, of a multiple emulsion are influenced. For this reason, multiple emulsions, the constituents and compositions of which were designed for a particular use, can generally not be applied to other uses by modifying or replacing the components thereof. This applies in particular when the essential constituents such as emulsifiers, electrolyte and the primary constituents of the various phases are affected.

However, the stability of the formulation is particularly significant in multiple emulsions that are used to apply sensitive active ingredients. This is because the active ingredient can only be effectively protected from harmful outside influences if it is entrapped in the innermost phase and this phase position is not changed by phase separation or inversion.

In addition to the phase stability of the emulsion, which represents a basic prerequisite for protecting an active ingredient that is encapsulated in the internal water phase W₂, however, other properties such as the diffusion properties are of importance. An encapsulated active ingredient, for example, may also degenerate when a “harmful substance” from outside finds its way into this phase, even if the active ingredient itself does not leave the internal phase.

In the case of a topical application, some active ingredients may already become damaged by natural constituents of the skin. This applies, for example, to substances that can degenerate by the enzymatic digestion of enzymes present on the skin. These include in particular peptides and nucleic acid-based compounds, such as those based on polynucleotides and oligonucleotides, which may be decomposed prematurely by ubiquitous nucleases or peptidases. The use of such compounds for treating diseases, however, has increasingly gained in importance over the course of the last few years. Antisense-oligonucleotides, but also ribozymes, RNAi, siRNA and in particular DNAzymes, can be used, for example, to prevent the formation of a protein in a target cell by suppressing the translation of the mRNA of this protein. DNAzymes are understood to mean single-stranded DNA molecules that exhibit enzymatic activity and, contrary to ribozymes, do not occur naturally. However, the problem is that these nucleotide active ingredients are generally very sensitive to environmental conditions and, without the aid of appropriate vehicles, frequently do not reach the sites of action without first degrading.

So far no multiple W/O/W emulsion is known from the prior art which has sufficient long-term stability to be able to lastingly ensure effective protection of such a sensitive active ingredient.

In addition, the problem with existing conventional W/O/W emulsions is that they are suitable for large-scale production only with great difficulty. Protection of the W/O/W emulsion against microbial contamination is also frequently not provided. As a result, they are suited only extremely conditionally, if at all, for applying one of the above-mentioned active ingredients.

It is thus the object of the invention to overcome these and further drawbacks of the prior art and to provide a multiple emulsion for applying an active ingredient. The multiple emulsion should in particular have high stability and offer effective protection of the active ingredient from outside influences, while allowing the emulsion to be protected against microbial contamination. Moreover, the emulsion should also be easy and cost-effective to produce on a large scale.

The main features of the invention are described in the characterizing part of claims 1, 17, 19 and 21. Embodiments are the subject matter of the dependent claims.

In a multiple emulsion for applying at least one pharmaceutical or cosmetic active ingredient, comprising an external water phase W₁, an oil phase O dispersed in the external water phase W₁, and an internal water phase W₂ dispersed in the oil phase O, wherein at least one electrolyte selected from the group of alkali metal and alkaline earth metal halides and sulfates and at least one hydrophilic active ingredient are provided in the internal water phase W₂, according to the invention:
the external water phase W₁ comprises a hydrophilic emulsifier, which is a polymer of ethylene oxide and propylene oxide, and
the oil phase O is formed of triacylglycerides and comprises a lipophilic emulsifier from the group of silicones.

The multiple emulsion according to the invention has particularly high stability due to the specific composition thereof. This applies both to the stability during production, up to and including on a large scale, and with respect to the long-term stability.

Another advantage is the simple composition of the multiple emulsion according to the invention, which allows uncomplicated and cost-effective production, starting from a small number of different components. Additionally, all the constituents of the emulsion according to the invention at the most have low irritation potential and are already established when it comes to the use thereof on the skin. Moreover, all the constituents are readily available in very high levels of purity.

The multiple emulsion according to the invention further offers particularly high protection for sensitive active ingredients entrapped in the internal water phase W₂ as a result of the special composition.

As a result of the specific combination of the ingredients that are provided according to the invention, in particular as a result of the combination of the emulsifiers that are provided together with an oil phase comprising triacylglycerides, a multiple W/O/W emulsion can be provided which has a comparatively high tolerance toward various other additives. This creates latitude in terms of optionally desired modifications and/or fine-tuning. It is of particularly great advantage in this regard that various preservatives for the protection against microbial contamination may be added without impairing the stability of the multiple emulsion.

In one important embodiment of the invention, the hydrophilic emulsifier is a poloxamer. Poloxamers are block polymers of ethylene oxide and propylene oxide. The use of Poloxamer 407 as the hydrophilic emulsifier is particularly preferred.

According to another important embodiment of the invention, the lipophilic emulsifier is a linear dimethicone having polyether and alkyl groups. Dimethicones that comprise polyether groups, such as polyethylene glycol (PEG) groups and/or polypropylene glycol (PPG) groups, and alkyl groups, as well as linear dimethicones are particularly preferred. Particularly good results are achieved when cetyl PEG/PPG-10/1 dimethicone (trade name: Degussa ABIL® EM 90) is the lipophilic emulsifier. By using the above-mentioned dimethicones as the lipophilic emulsifier, the drop size of the primary w/o emulsion can be adjusted very well and the stability of the emulsion can be considerably improved. In addition, use of the above-mentioned dimethicones as the lipophilic emulsifier also allows the drop size of the multiple w/o/w emulsion to be adjusted well over a wide range, as well as the stability of the emulsion. In addition, the drop size of such emulsions no longer has any influence on the long-term stability. Moreover, greater tolerance with respect to necessary additives can be achieved.

It may also be advantageous if the triacylglycerides provided according to the invention are esters of saturated fatty acids, preferably esters of medium-chain saturated fatty acids. Among these, esters of fatty acids having 4 to 20 carbon atoms, and notably esters of fatty acids having 6 to 14 carbon atoms, have proven to be advantageous.

Particularly stable W/O/W emulsions can be obtained with triglycerides that have one of the following chain length mixes:

1) Chain length mix (gas chromatography):
C chain length distribution C 10 20.0-50.0%
C chain length distribution C 12<=3.0%
C chain length distribution C 14<=1.0%
C chain length distribution C 6<=2.0%
C chain length distribution C 8 50.0-80.0%

2) Chain length mix (gas chromatography):
caproic acid C6<=2.0% 0.1%
caprylic acid C8 50.0 to 80.0% 55.7%
caprinic acid C10 20.0 to 50.0% 43.3%
lauric acid C12<=3.0% 0.6%
myristic acid C14<=1.0% 0.0%

In one preferred embodiment of the invention, the electrolyte is NaCl, MgSO₄ or a mixture of NaCl and MgSO₄.

According to another embodiment of the invention, the hydrophilic active ingredient is MgSO₄. MgSO₄ advantageously affects the skin complexion. A multiple emulsion according to the invention comprising this active ingredient is therefore suitable for supportive skin care and the treatment of neurodermatitis.

Another important embodiment further provides for the hydrophilic active ingredient to be an oligonucleotide. For example, such an oligonucleotide can be a DNAzyme, in particular the 10-23 DNAzyme, which includes sequence SEQ ID NO: 149 ggctagctacaacga as the catalytic domain. The catalytic domain is flanked by a right and a left substrate binding domain. This domain can preferably be directed against the mRNA sequence of a factor that is involved in one of the signal transduction pathways of chronic inflammatory diseases. It is conceivable, for example, without being exhaustive, that the right and left substrate binding domains may bind to an mRNA that codes for one of the following factors: Src kinase, Tec kinase, Rlk, Itk, RIBP (Rlk/ltk-binding protein), PLCg (phospholipase Cy1), MAP kinase, ERK, JNK, P38, MKK, Rac2, GADD45, SOCS, CIS, JAK, NF-AT interacting protein.

DNAzymes, which preferably are used as active ingredients in the multiple emulsion according to the invention, thus include the general sequence SEQ ID. NO: 150: nnnnnnnnnggctagctacaacgannnnnnnnn. The right and left substrate binding domains in the preferred exemplary embodiments are at least nine bases long, respectively. However, shorter or longer sequences are also conceivable, of course.

Substrate binding domains that bind the mRNA of proteins GATA-3 and T-bet are particularly preferred. Inhibition of the translation of these proteins results in a suppression of the response of T2 cells and thus suppresses inflammatory diseases.

The hydrophilic active ingredient is particularly preferably an oligonucleotide having a sequence according to one of the sequences SEQ ID NO: 1 to SEQ ID NO: 148 or of the above-described SEQ ID NO: 150. Of course it is also conceivable to simultaneously use several of the described oligonucleotides. The catalytic domain of the DNAzymes is shown in lower case letters, the substrate binding domains are shown in upper case letters. Sequences SEQ ID NO: 1 to SEQ ID NO: 71 bind the mRNA of GATA-3. Sequences SEQ ID NO: 72 to SEQ ID NO: 148 bind the mRNA of T-bet.

The use of an oligonucleotide having a sequence SEQ ID NO: 40, SEQ ID NO: 139 or SEQ ID NO: 140 as the hydrophilic active ingredient is particularly preferred.

So as to ensure protection of the emulsion against microbial contamination, it may be provided that the multiple emulsion includes one or more preservatives. Preferred preservatives are potassium sorbate, potassium sorbate with citric acid, sorbic acid, benzoic acid, polyols such as preferably pentylene glycol, butylene glycol and/or propylene glycol, parabens such as preferably methylparaben and/or propylparaben, as well as phenoxyethanol. The preservative is preferably provided in the external water phase W₁ and/or in the internal water phase W₂, preferably in a content of >0 to 20% by weight based on the water.

One important embodiment additionally provides for the drop size of the dispersed oil phase to be 1 ∥m to 100 μm, preferably 5 μm to 60 μm, and particularly preferably 10 μm to 30 μm.

Depending on use, it may be advantageous if a saturated hydrocarbon is present in the oil phase. The preferably saturated hydrocarbon is paraffin, in particular viscous paraffin.

Particularly advantageous emulsions may be obtained when the hydrophilic emulsifier is present in the external water phase W₁ in a content of 0.1 to 5% by weight, preferably 0.3 to 3% by weight, particularly preferably 0.5 to 1.5% by weight, and most preferably in a content of 0.8% by weight.

In addition, the stability of the multiple emulsion is particularly high when the lipophilic emulsifier, based on a W/O primary emulsion consisting of the oil phase O and the internal water phase W₂, is present in a content of 0.1 to 6% by weight, preferably 0.5 to 4% by weight, particularly preferably 1 to 3% by weight, and most preferably in a content of 3% by weight.

With respect to the triglycerides, it is advantageous when the triglycerides, based on a W/O primary emulsion consisting of the oil phase O and the internal water phase W₂, are present in a content of 5 to 30% by weight, preferably 0 to 25% by weight, particularly preferably 15 to 20% by weight, and most preferably in a content of 17% by weight.

In a preferred embodiment, the electrolyte, based on a W/O primary emulsion consisting of the oil phase O and the internal water phase W₂, is present in a content of 0.1 to 3% by weight, particularly preferably 0.3 to 1.5% by weight, and most preferably in a content of 0.7% by weight. It may have an advantageous effect if the amount of electrolyte is adapted to the amount of active ingredient.

Based on the total mass of the multiple emulsion, the hydrophilic active ingredient is present in a content of 0.01 to 10% by weight, particularly preferably 0.1 to 5, and most preferably in a content of 0.4% by weight.

Within this meaning, a preferred multiple emulsion according to the invention is one in which:
the hydrophilic emulsifier in the external water phase W₁ has a content of 0.1 to 5% by weight; and
the lipophilic emulsifier, based on a W/O primary emulsion consisting of the oil phase O and the internal water phase W₂, is present in a content of 0.1 to 6% by weight; and
the triglycerides, based on the W/O primary emulsion, are present in a content of 5 to 30% by weight; and
the electrolyte, based on a W/O primary emulsion consisting of the oil phase O and the internal water phase W₂, is present in a content of 0.1 to 3% by weight; and
the hydrophilic active ingredient, based on the total mass of the multiple emulsion, is present in a content of 0.01 to 10% by weight; and
a preservative is present in the external water phase W₁ and/or in the internal water phase W₂ in a content of 0 to 20% by weight.

High stability and effective protection of the active ingredient are achieved above all with multiple emulsions according to the invention in which

based on a W/O primary emulsion
3% by weight lipophilic emulsifier, in particular Abil EM 90;
17% by weight oil, in particular medium-chain triglycerides;
0.7% by weight electrolyte, in particular MgSO₄*7H₂O;
water to make 100% by weight
   are present, wherein based on the multiple emulsion
80% by weight W/O emulsion;
0.8% by weight hydrophilic emulsifier, in particular poloxamer;
water up to 100% by weight are present,
   and wherein the finished multiple W/O/W emulsion comprises 0.4% by weight hydrophilic active ingredient, in particular a sodium salt of one of the above-mentioned DNAzymes.

Additionally, the invention provides for the use of a multiple emulsion according to the invention for the production of a pharmaceutical, a medicinal product or a cosmetic. It is particularly advantageous here when the multiple emulsion is used as a drug vehicle. The drug may be a pharmaceutical or cosmetic active ingredient. In this way, pharmaceuticals can be provided which are suitable for administering highly sensitive and highly specific active ingredients, for example by topical application or in another expedient form. The pharmaceuticals thus composed are particularly well-suited for treating diseases that can be attributed to malfunctions of very specific molecular-biological factors, for example GATA-3 or one of the other above-mentioned factors.

In a further aspect, the invention thus also relates to a pharmaceutical comprising a multiple emulsion according to the invention for topical use, or for the application of the active ingredient by way of mucous membranes, or for the intraperitoneal application of the active ingredient, wherein it is likewise particularly advantageous here when the multiple emulsion is a drug vehicle. One preferred embodiment relates to a pharmaceutical for treating an inflammatory diseases.

Further features, details and advantages of the invention will be apparent from the wording of the claims and from the following description of exemplary embodiments and the figures.

In the following figures and sequences:

| | |
|---|---|
| SEQ ID NO: 1 to | show exemplary embodiments for the 10-23 |
| SEQ ID NO: 148 | DNAzyme binding to GATA-3 or T-bet; |
| SEQ ID NO: 149 | is the catalytic domain of the 10-23 DNAzyme; |
| SEQ ID NO: 150 | is the DNAzyme comprising the catalytic domain |
| | 10-23 DNAzyme; |

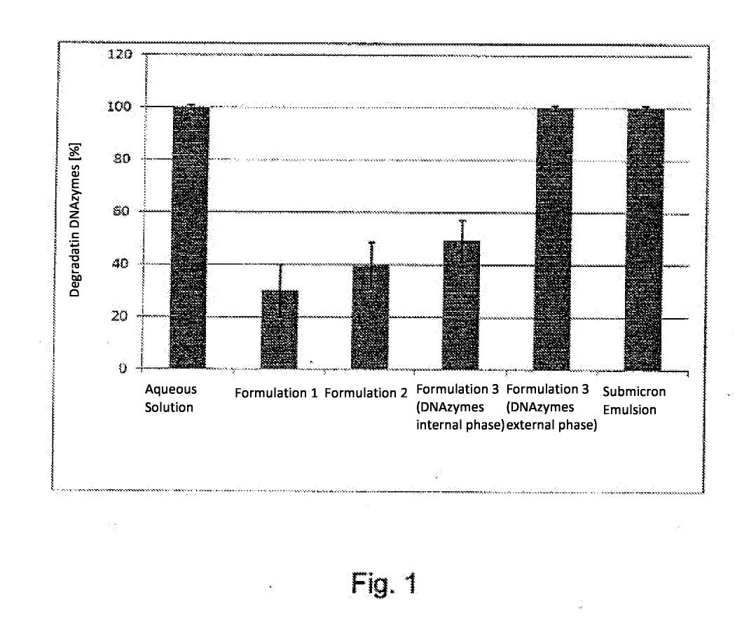

FIG. 1 shows measuring data for the verification of the protective function of an emulsion according to the invention;

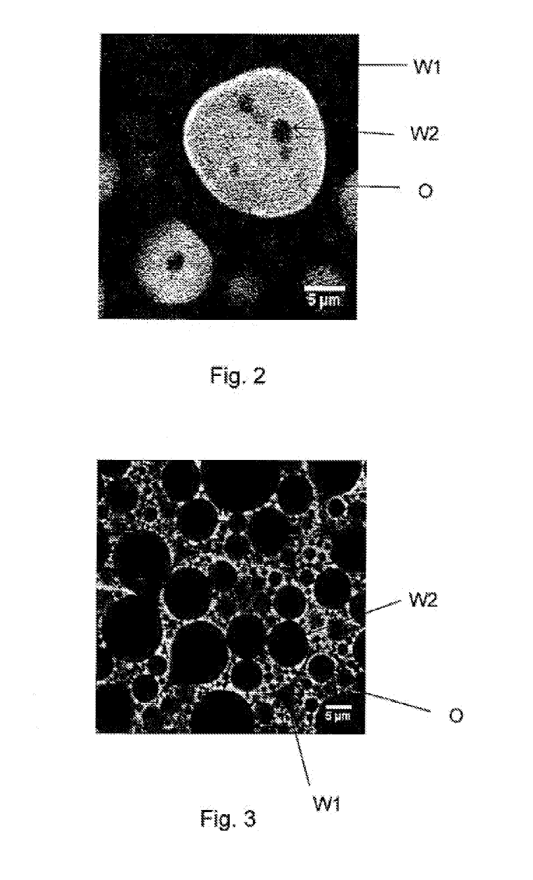

FIG. 2 shows the internal structure of a multiple comparison emulsion; and

FIG. 3 shows the internal structure of a multiple emulsion from the basic formulation WOW_{—}1 (for composition see exemplary embodiments, item 2)).

### EXEMPLARY EMBODIMENTS

The following ingredients were used in the exemplary embodiments shown below, unless indicated otherwise:

| Ingredients | Manufacturer's Name | Manufacturer |
|---|---|---|
| Internal water phase W₂: | | |
| Distilled water | magnesium sulfate | Fagron |
| MgSO₄•7H₂O | heptahydrate | Biospring |
| DNAzyme Na salt | | Fagron |
| Potassium sorbate | | Fabron |
| Citric acid | | |
| Oil phase O: | | |
| Medium-chain triglycerides | Abil EM 90 | Fabron |
| Cetyl PEG/PPG-10/1 | | Evonik |
| Dimethicones | | |
| External water phase W₂: | | |
| Distilled water | | Fagron |
| Potassium sorbate | | Fagron |
| Citric acid | | Fagron |
| Poloxamer 407 | | |

### 1) General Production Method

The emulsions described below were prepared according to the following general production method:

First, three phases, A, B and C are produced. For the production of phase A, the constituents that are provided for the internal water phase W₂ of the emulsion are mixed, which is to say, for example, distilled water, magnesium sulfate heptahydrate, DNAzyme, potassium sorbate and citric acid in the amounts indicated in the lists below. For the production of phase B, the constituents that are provided for the oil phase O of the emulsion are mixed, which is to say, for example, medium-chain triglycerides and Abil® EM 90 in the amounts indicated in the lists below. For the production of phase C, the constituents that are provided for the external water phase C are mixed, which is to say, for example, distilled water, potassium sorbate, citric acid and Poloxamer 407 in the amounts indicated in the lists below.

Thereafter, phase A and phase B are heated individually to approximately 60 to 70° C. Phase A is then added to Phase B and homogenized for approximately 3 minutes (homogenizer: approximately 11500 rpm), wherein the W/O primary emulsion is formed. The W/O primary emulsion is cooled to room temperature while stirring (stirrer: approximately 500 rpm) and is then mixed with phase C. After phase C has been added, the mixture is stirred (stirrer: approximately 1200 rpm) and/or homogenized until a uniform emulsion has been obtained (duration approximately 10 minutes).

The described production method is used, for example, to produce multiple emulsions in which:
the hydrophilic emulsifier in the external water phase W₁ has a content of 0.1 to 5% by weight; and
the lipophilic emulsifier, based on a W/O primary emulsion consisting of the oil phase O and the internal water phase W₂, is present in a content of 0.1 to 6% by weight; and
the triglycerides, based on the W/O primary emulsion, are present in a content of 5 to 30% by weight; and
the electrolyte, based on a W/O primary emulsion consisting of the oil phase O and the internal water phase W₂, is present in a content of 0.1 to 3% by weight; and
the hydrophilic active ingredient, based on the total mass of the multiple emulsion, is present in a content of 0.01 to 10% by weight; and
a preservative is present in the external water phase W₁ and/or in the internal water phase W₂ in a content of 0 to 20% by weight.

In particular formulations were produced by way of the production method in which:

based on a W/O primary emulsion
3% by weight lipophilic emulsifier, in particular Abil EM 90;
17% by weight oil, in particular medium-chain triglycerides;
0.7% by weight electrolyte, in particular MgSO₄*7 H₂O;
water to make 100% by weight
   are present, wherein based on the multiple emulsion
80% by weight W/O emulsion;
0.8% by weight hydrophilic emulsifier, in particular poloxamer;
water to make 100% by weight are present,
   and wherein the finished multiple W/O/W emulsion comprises 0.4% by weight hydrophilic active ingredient,
   in particular a sodium salt of one of the DNAzymes mentioned below in item 4) of the exemplary embodiments.

The production of the following multiple emulsions is mentioned by way of example:

### WOW_A1 (100 g Scale)

| | | | | | |
|---|---|---|---|---|---|
| A | MgSO₄•7H₂O | 0.56 | g | 0.56% | by weight^{a} |
| | Distilled water | 63.307 | g | 63.307% | by weight^{a} |
| | Potassium sorbate | 0.089 | g | 0.089% | by weight^{a} |
| | Citric acid | 0.044 | g | 0.044% | by weight^{a} |
| B | Medium-chain triglycerides | 13.6 | g | 13.6% | by weight^{a} |
| | Cetyl PEG/PPG-10/1 | 2.4 | g | 2.4% | by weight^{a} |
| | dimethicone | | | | |
| C | Distilled water | 9.160 | g | 19.160% | by weight^{a} |
| | Potassium sorbate | 0.027 | g | 0.027% | by weight^{a} |
| | Citric acid | 0.013 | g | 0.013% | by weight^{a} |
| | Poloxamer 407 | 0.8 | g | 0.8% | by weight a |

^{a}% by weight, based on the total mass of the multiple emulsion

### WOW_A2 (12000 g Scale)

| | | | | | |
|---|---|---|---|---|---|
| A | MgSO₄•7H₂O | 7.2 | g | 0.56% | by weight^{a} |
| | Distilled water | 7596.813 | g | 63.307% | by weight^{a} |
| | Potassium sorbate | 10.658 | g | 0.089% | by weight^{a} |
| | Citric acid | 5.329 | g | 0.044% | by weight^{a} |
| B | Medium-chain triglycerides | 1632 | g | 13.6% | by weight^{a} |
| | Cetyl PEG/PPG-10/1 | 288 | g | 2.4% | by weight^{a} |
| | dimethicone | | | | |
| C | Distilled water | 2299.162 | g | 19.160% | by weight^{a} |
| | Potassium sorbate | 3.226 | g | 0.027% | by weight^{a} |
| | Citric acid | 1.612 | g | 0.013% | by weight^{a} |
| | Poloxamer 407 | 96 | g | 0.8% | by weight^{a} |

^{a}% by weight, based on the total mass of the multiple emulsion

### WOW_A3 (100 g Scale)

| | | | | | |
|---|---|---|---|---|---|
| A | MgSO₄•7H₂O | 0.56 | g | 0.56% | by weight^{a} |
| | Distilled water | 62.907 | g | 62.907% | by weight^{a} |
| | Na salt of DNAzyme | 0.4 | g | 0.4% | by weight^{a} |
| | Potassium sorbate | 0.089 | g | 0.089% | by weight^{a} |
| | Citric acid | 0.044 | g | 0.044% | by weight^{a} |
| B | Medium-chain triglycerides | 13.6 | g | 13.6% | by weight^{a} |
| | Cetyl PEG/PPG-10/1 | 2.4 | g | 2.4% | by weight^{a} |
| | dimethicone | | | | |
| C | Distilled water | 19.160 | g | 19.160% | by weight^{a} |
| | Potassium sorbate | 0.027 | g | 0.027% | by weight^{a} |
| | Citric acid | 0.013 | g | 0.013% | by weight^{a} |
| | Poloxamer 407 | 0.8 | g | 0.8% | by weight^{a} |

^{a}% by weight, based on the total mass of the multiple emulsion

### 2) Basic Formulation of a Multiple Emulsion WOW_{—}1 According to the Invention

TABLE 1
Composition of the W/O/W emulsion WOW_1

| | Ingredients | [w/w] % |
|---|---|---|
| WO: | Abil EM 90 | 3 |
| | (lipophilic emulsifier) | |
| | Medium-chain triglycerides | 17 |
| | (oil) | |
| | MgSO4 * 7H2O | 0.7 |
| | (electrolyte and/or active | |
| | ingredient) | |
| | H2O | 79.3 |
| WOW: | WO | 80 |
| | Poloxamer 407 | 0.8 |
| | (hydrophilic emulsifier) | |
| | H2O | 19.2 |

In the embodiment of the invention shown, both the electrolyte and the hydrophilic active ingredient are MgSO₄. A dimethicone, which is to say Abil EM 90, is used as the lipophilic emulsifier. Triacylglycerides, which is to say medium-chain triglycerides, form the oil. The hydrophilic emulsifier is a polymer of ethylene oxide and propylene oxide, which is to say Poloxamer 407.

The basic formulation WOW_{—}1 is produced according to the above-described general production method.

The drop size of the dispersed oil phase of the multiple emulsion WOW_{—}1 is 30 μm; the viscosity is 1.2 Pa s.

When long-term stability measurements are carried out in a time period of 9 months, for example, the emulsion remains stable during this time. It exhibits no significant change in drop size and viscosity.

### 3) Influence of the Composition on the Stability

### 3.1) Influence of the Lipophilic Emulsifier on the Stability of a W/O/W Emulsion

So as to analyze the influence of the lipophilic emulsifier, a multiple emulsion WOW_{—}1 according to exemplary embodiment 1) is produced, wherein in addition to Abil 90 EM, sorbitan monooleate is added to the primary emulsion as a further lipophilic emulsifier.

Changing the lipophilic emulsifier results in a destabilization of the W/O/W emulsion.

### 3. 2) Additional Modifications

| Ingredients | Drop size | Viscosity | Phase stability |
|---|---|---|---|
| Basic formulation | +++ | +++ | +++ |
| M1: | + | − | + |
| WOW 250 | | | |
| W₂: MgSO4 | | | |
| Oil phase: Abil EM 90, medium- | | | |
| chain triglycerides, decyl oleate, | | | |
| lecithin | | | |
| W₁: Poloxamer 407 | | | |
| Preservative: butylene glycol, | | | |
| glycerin | | | |
| M2: | ++ | ++ | +++ |
| WOW 251 | | | |
| W₂: MgSO4 | | | |
| Oil phase: Abil EM 90, medium- | | | |
| chain triglycerides, decyl oleate, | | | |
| sorbitan monooleates | | | |
| W₁: Poloxamer 407 | | | |
| Preservative: butylene glycol, | | | |
| glycerin | | | |
| M3: | + | − | + |
| WOW 255 | | | |
| W₂: MgSO4, NaCl | | | |
| Oil phase: Abil EM 90, viscous | | | |
| paraffin, lecithin, sorbitan | | | |
| monooleates | | | |
| W₁: Steareth-20 | | | |
| Preservative: benzoic acid | | | |
| M4: | +++ | + | +++ |
| WOW 255 | | | |
| W₂: MgSO4 | | | |
| Oil phase: Abil EM 90, medium- | | | |
| chain triglycerides | | | |
| W₁: Steareth-20 | | | |
| Preservative: butylene glycol | | | |
| M5: | + | + | +++ |
| WOW 256 | | | |
| W₂: MgSO4 | | | |
| Oil phase: Abil EM 90, viscous | | | |
| paraffin, lecithin | | | |
| W₁: Poloxamer 407 | | | |
| Preservative: butylene glycol | | | |
| M6: | + | + | +++ |
| WOW 257 | | | |
| W₂: MgSO4 | | | |
| Oil phase: Abil EM 90, medium- | | | |
| chain triglycerides | | | |
| W₁: Steareth-20 | | | |
| Preservative: butylene glycol, | | | |
| glycerin | | | |
| M7: | ++ | ++ | +++ |
| WOW 260 | | | |
| W₂: MgSO4 | | | |
| Oil phase: Abil EM 90, medium- | | | |
| chain triglycerides, decyl oleates | | | |
| (not Ph. Eur.) | | | |
| W₁: Poloxamer 407 | | | |
| Preservative: potassium sorbate, | | | |
| glycerin | | | |

Evaluation key:
+++ Very good
++ good
+ satisfactory
− poor

As the test results show, only emulsions M1 to M7 deliver good results. This demonstrates that the emulsion according to the invention exhibits high tolerance toward various additives.

### 4) Protective Action of the Multiple Emulsion

For verification of the protective action of the multiple emulsion, various formulations that comprise a DNAzyme, to mention one example of a nucleotide-based sensitive active ingredient, are mixed with a nuclease solution.

The test results are shown in the diagram in FIG. 1.

Test Execution:

20 mg of the respective formulation is mixed with 1 ml of a DNase I solution (skin's own DNases). Following an incubation period of 1 minute, the mixture is shaken lightly for 10 minutes in a thermal shaker at a temperature of 99° C. so as to stop the activity of the DNase. So as to break the emulsion, the batch is incubated in an ultrasonic bath at 50° C. for 10 minutes. The batch is filtered through a 0.45 μm syringe filter and the intact active ingredient amount is determined by way of HPLC analysis.

All formulations comprise 0.4% of a DNAzyme (Na salt). Formulations 1, 2 (columns 2 and 3) comprise the DNAzyme in the internal water phase W₂. For comparison, DNAzyme was added to formulation 3 once in the internal water phase W₂ (column 4) and once in the external water phase W₁ (column 5). Column 1 shows the test result obtained with a simple aqueous solution of the DNAzyme, and column 6 shows the test result obtained with a submicron emulsion.

The DNAzyme used in each case is a DNAzyme having one of the following sequences:

SEQ ID. NO 150: nnnnnnnnnggctagctacaacgannnnnnnnn. The right and left substrate binding domains in the preferred exemplary embodiments are at least nine bases long, respectively.

hgd1
SEQ ID No: 1
5′-TCGGTCAGAggctagctacaacgaTGCGTTGCT-3′
hgd2
SEQ ID No: 2
5′-GGCGTACGAggctagctacaacgaCTGCTCGGT-3′
hgd3
SEQ ID No: 3
5′-GGCGGCGTAggctagctacaacgaGACCTGCTC-3′
hgd4
SEQ ID No: 4
5′-CTCGGGTCAggctagctacaacgaCTGGGTAGC-3′
hgd5
SEQ ID No: 5
5′-TCCTCTGCAggctagctacaacgaCGGGGTCCT-3′
hgd6
SEQ ID No: 6
5′-ACTCTGCAAggctagctacaacgaTCTGCGAGC-3′
hgd7
SEQ ID No: 7
5′-GGGCGACGAggctagctacaacgaTCTGCAATT-3′
hgd8
SEQ ID No: 8
5′-AAGGGGCGAggctagctacaacgaGACTCTGCA-3′
hgd9
SEQ ID No: 9
5′-AAAACGGGAggctagctacaacgaCAGGTTGTA-3′
hgd10
SEQ ID No: 10
5′-AGAATAAAAggctagctacaacgaGGGACCAGG-3′
hgd11
SEQ ID No: 11
5′-ATGGCAGAAggctagctacaacgaAAAACGGGA-3′
hgd12
SEQ ID No: 12
5′-AACTGGGTAggctagctacaacgaGGCAGAATA-3′
hgd13
SEQ ID No: 13
5′-ATCCAAAAAggctagctacaacgaTGGGTATGG-3′
hgd14
SEQ ID No: 14
5′-AGGGGAAGAggctagctacaacgaAAAAATCCA-3′
hgd15
SEQ ID No: 15
5′-TTTTAAAAAggctagctacaacgaTATCTTGGA-3′
hgd16
SEQ ID No: 16
5′-GTGGGGGGAggctagctacaacgaGGGAAGGCT-3′
hgd17
SEQ ID No: 17
5′-GTTGAATGAggctagctacaacgaTTGCTTTCG-3′
hgd18
SEQ ID No: 18
5′-GTCGTTGAAggctagctacaacgaGATTTGCTT-3′
hgd19
SEQ ID No: 19
5′-GGCCCGGAAggctagctacaacgaCCGCGCGCG-3′
hgd20
SEQ ID No: 20
5′-TCACCTCCAggctagctacaacgaGGCCTCGGC-3′
hgd21
SEQ ID No: 21
5′-CCGCCGTCAggctagctacaacgaCTCCATGGC-3′
hgd22
SEQ ID No: 22
5′-GGTGGCTCAggctagctacaacgaCCAGCGCGG-3′
hgd23
SEQ ID No: 23
5′-CGTTGAGCAggctagctacaacgaGGCGGGGTG-3′
hgd24
SEQ ID No: 24
5′-CCGCGTCCAggctagctacaacgaGTAGGAGTG-3′
hgd25
SEQ ID No: 25
5′-CAGCGGGTAggctagctacaacgaTGCGCCGCG-3′
hgd26
SEQ ID No: 26
5′-GCACATCCAggctagctacaacgaCTCCTCCGG-3′
hgd27
SEQ ID No: 27
5′-AAAAGCACAggctagctacaacgaCCACCTCCT-3′
hgd28
SEQ ID No: 28
5′-TAAAAAGCAggctagctacaacgaATCCACCTC-3′
hgd29
SEQ ID No: 29
5′-GACCGTCGAggctagctacaacgaGTTAAAAAG-3′
hgd30
SEQ ID No: 30
5′-TTGCCTTGAggctagctacaacgaCGTCGATGT-3′
hgd31
SEQ ID No: 31
5′-AGGGCGGGAggctagctacaacgaGTGGTTGCC-3′
hgd32
SEQ ID No: 32
5′-TGGCCCTGAggctagctacaacgaCGAGTTTCC-3′
hgd33
SEQ ID No: 33
5′-ACCTCTGCAggctagctacaacgaCGTGGCCCT-3′
hgd34
SEQ ID No: 34
5′-CGGAGGGTAggctagctacaacgaCTCTGCACC-3′
hgd35
SEQ ID No: 35
5′-GGCGGCACAggctagctacaacgaCTGGCTCCC-3′
hgd36
SEQ ID No: 36
5′-CGGGCGGCAggctagctacaacgaACCTGGCTC-3′
hgd37
SEQ ID No: 37
5′-AGGGATCCAggctagctacaacgaGAAGCAGAG-3′
hgd38
SEQ ID No: 38
5′-GGGTAGGGAggctagctacaacgaCCATGAAGC-3′
hgd39
SEQ ID No: 39
5′-GGGCTGAGAggctagctacaacgaTCCAGGGGG-3′
hgd40
SEQ ID No: 40
5′-GTGGATGGAggctagctacaacgaGTCTTGGAG-3′
hgd41
SEQ ID No: 41
5′-CGTGGTGGAggctagctacaacgaGGACGTCTT-3′
hgd42
SEQ ID No: 42
5′-GGGGGTAGAggctagctacaacgaGGAGAGGGG-3′
hgd43
SEQ ID No: 43
5′-GGAGGAGGAggctagctacaacgaGAGGCCGGG-3′
hgd44
SEQ ID No: 44
5′-GCCCCCCGAggctagctacaacgaAAGGAGGAG-3′
hgd45
SEQ ID No: 45
5′-CCGGGGAGAggctagctacaacgaGTCCTTCGG-3′
hgd46
SEQ ID No: 46
5′-GGACAGCGAggctagctacaacgaGGGTCCGGG-3′
hgd47
SEQ ID No: 47
5′-TGGGGTGGAggctagctacaacgaAGCGATGGG-3′
hgd48
SEQ ID No: 48
5′-CTTGAGGCAggctagctacaacgaTCTTTCTCG-3′
hgd49
SEQ ID No: 49
5′-CACCTGGTAggctagctacaacgaTTGAGGCAC-3′
hgd50
SEQ ID No: 50
5′-GCAGGGGCAggctagctacaacgaCTGGTACTT-3′
hgd51
SEQ ID No: 51
5′-CCAGCTTCAggctagctacaacgaGCTGTCGGG-3′
hgd52
SEQ ID No: 52
5′-GTGGGACGAggctagctacaacgaTCCAGCTTC-3′
hgd53
SEQ ID No: 53
5′-GGAGTGGGAggctagctacaacgaGACTCCAGC-3′
hgd54
SEQ ID No: 54
5′-ATGCTGCCAggctagctacaacgaGGGAGTGGG-3′
hgd55
SEQ ID No: 55
5′-GGGCGGTCAggctagctacaacgaGCTGCCACG-3′
hgd56
SEQ ID No: 56
5′-GAGGCTCCAggctagctacaacgaCCAGGGCGG-3′
hgd57
SEQ ID No: 57
5′-GTGGGTCGAggctagctacaacgaGAGGAGGCT-3′
hgd58
SEQ ID No: 58
5′-AGGTGGTGAggctagctacaacgaGGGGTGGTG-3′
hgd59
SEQ ID No: 59
5′-ACTCGGGCAggctagctacaacgaGTAGGGCGG-3′
hgd60
SEQ ID No: 60
5′-GGAGCTGTAggctagctacaacgaTCGGGCACG-3′
hgd61
SEQ ID No: 61
5′-GGACTTGCAggctagctacaacgaCCGAAGCCG-3′
hgd62
SEQ ID No: 62
5′-GGGCCTGGAggctagctacaacgaTTGCATCCG-3′
hgd63
SEQ ID No: 63
5′-TGTGCTGGAggctagctacaacgaCGGGCCTTG-3′
hgd64
SEQ ID No: 64
5′-GTTCACACAggctagctacaacgaTCCCTGCCT-3′
hgd65
SEQ ID No: 65
5′-CAGTTCACAggctagctacaacgaACTCCCTGC-3′
hgd66
SEQ ID No: 66
5′-CACAGTTCAggctagctacaacgaACACTCCCT-3′
hgd67
SEQ ID No: 67
5′-GTTGCCCCAggctagctacaacgaAGTTCACAC-3′
hgd68
SEQ ID No: 68
5′-TCGCCGCCAggctagctacaacgaAGTGGGGTC-3′
hgd69
SEQ ID No: 69
5′-CCCGTGCCAggctagctacaacgaCTCGCCGCC-3′
hgd70
SEQ ID No: 70
5′-GGCGTTGCAggctagctacaacgaAGGTAGTGT-3′
td1
SEQ ID No: 71
5′-TGGCTTCTAggctagctacaacgaGCCCTCGTC-3′
td2
SEQ ID No: 72
5′-GGGCTCTGAggctagctacaacgaGCCTGGCTT-3′
td3
SEQ ID No: 73
5′-GGGACCCCAggctagctacaacgaCGGAGCCCG-3′
td4
SEQ ID No: 74
5′-GGTGGGGGAggctagctacaacgaCCCACCGGA-3′
td5
SEQ ID No: 75
5′-GGCGGGGGAggctagctacaacgaCCGAGGGCC-3′
td6
SEQ ID No: 76
5′-GGGCTGGGAggctagctacaacgaGGGCAGGGA-3′
td7
SEQ ID No: 77
5′-CGTCGAGGAggctagctacaacgaCCGCCCCTC-3′
td8
SEQ ID No: 78
5′-GGGCTGGCAggctagctacaacgaCTTCCCGTA-3′
td9
SEQ ID No: 79
5′-CGATGCCCAggctagctacaacgaCCGGGGCGG-3′
td10
SEQ ID No: 80
5′-GCTCCACGAggctagctacaacgaGCCCATCCG-3′
td11
SEQ ID No: 81
5′-CCGGCTCCAggctagctacaacgaGATGCCCAT-3′
td12
SEQ ID No: 82
5′-TCTCCGCAAggctagctacaacgaCCGGCTCCA-3′
td13
SEQ ID No: 83
5′-CCGTCAGCAggctagctacaacgaGTCTCCGCA-3′
td14
SEQ ID No: 84
5′-TCCCCGGCAggctagctacaacgaCGGCTCGGT-3′
td15
SEQ ID No: 85
5′-CCCCCGCGAggctagctacaacgaGCTCGTCCG-3′
td16
SEQ ID No: 86
5′-GTAGGGAGAggctagctacaacgaCCCAGGCTG-3′
td17
SEQ ID No: 87
5′-GGGCGGGCAggctagctacaacgaCAAGGCGCC-3′
td18
SEQ ID No: 88
5′-CGGGAAGGAggctagctacaacgaTCGCCCGCG-3′
td19
SEQ ID No: 89
5′-TAGTCCTCAggctagctacaacgaGCGGCCCCG-3′
td20
SEQ ID No: 90
5′-TCCCCGACAggctagctacaacgaCTCCAGTCC-3′
td21
SEQ ID No: 91
5′-TTTCCCCGAggctagctacaacgaACCTCCAGT-3′
td22
SEQ ID No: 92
5′-TGAGCGCGAggctagctacaacgaCCTCAGTTT-3′
td23
SEQ ID No: 93
5′-GGACCACAAggctagctacaacgaAGGTGGTTG-3′
td24
SEQ ID No: 94
5′-CTTGGACCAggctagctacaacgaAACAGGTGG-3′
td25
SEQ ID No: 95
5′-AAACTTGGAggctagctacaacgaCACAACAGG-3′
td26
SEQ ID No: 96
5′-CTGATTAAAggctagctacaacgaTTGGACCAC-3′
td27
SEQ ID No: 97
5′-TGGTGCTGAggctagctacaacgaTAAACTTGG-3′
td28
SEQ ID No: 98
5′-TGATGATCAggctagctacaacgaCTCTGTCTG-3′
td29
SEQ ID No: 99
5′-TGGTGATGAggctagctacaacgaCATCTCTGT-3′
td30
SEQ ID No: 100
5′-GCTTGGTGAggctagctacaacgaGATCATCTC-3′
td31
SEQ ID No: 101
5′-ATGGGAACAggctagctacaacgaCCGCCGTCC-3′
td32
SEQ ID No: 102
5′-GAATGGGAAggctagctacaacgaATCCGCCGT-3′
td33
SEQ ID No: 103
5′-TGACAGGAAggctagctacaacgaGGGAACATC-3′
td34
SEQ ID No: 104
5′-AGTAAATGAggctagctacaacgaAGGAATGGG-3′
td35
SEQ ID No: 105
5′-CACAGTAAAggctagctacaacgaGACAGGAAT-3′
td36
SEQ ID No: 106
5′-GCCCGGCCAggctagctacaacgaAGTAAATGA-3′
td37
SEQ ID No: 107
5′-CCACAAACAggctagctacaacgaCCTGTAGTG-3′
td38
SEQ ID No: 108
5′-GTCCACAAAggctagctacaacgaATCCTGTAG-3′
td39
SEQ ID No: 109
5′-CCACGTCCAggctagctacaacgaAAACATCCT-3′
td40
SEQ ID No: 110
5′-CCAAGACCAggctagctacaacgaGTCCACAAA-3′
td41
SEQ ID No: 111
5′-CCACCAAGAggctagctacaacgaCACGTCCAC-3′
td42
SEQ ID No: 112
5′-GCTGGTCCAggctagctacaacgaCAAGACCAC-3′
td43
SEQ ID No: 113
5′-GCTCTGGTAggctagctacaacgaCGCCAGTGG-3′
td44
SEQ ID No: 114
5′-CTGCACCCAggctagctacaacgaTTGCCGCTC-3′
td45
SEQ ID No: 115
5′-CACACTGCAggctagctacaacgaCCACTTGCC-3′
td46
SEQ ID No: 116
5′-CTTTCCACAggctagctacaacgaTGCACCCAC-3′
td47
SEQ ID No: 117
5′-GCCTTTCCAggctagctacaacgaACTGCACCC-3′
td48
SEQ ID No: 118
5′-TTCCTGGCAggctagctacaacgaGCTGCCCTC-3′
td49
SEQ ID No: 119
5′-GTGGACGTAggctagctacaacgaAGGCGGTTT-3′
td50
SEQ ID No: 120
5′-CCGGGTGGAggctagctacaacgaGTACAGGCG-3′
td51
SEQ ID No: 121
5′-CCTGGCGCAggctagctacaacgaCCAGTGCGC-3′
td52
SEQ ID No: 122
5′-CAAATGAAAggctagctacaacgaTTCCTGGCG-3′
td53
SEQ ID No: 123
5′-TTTCCCAAAggctagctacaacgaGAAACTTCC-3′
td54
SEQ ID No: 124
5′-ATTGTTGGAggctagctacaacgaGCCCCCTTG-3′
td55
SEQ ID No: 125
5′-TGGGTCACAggctagctacaacgaTGTTGGACG-3′
td56
SEQ ID No: 126
5′-TCTGGGTCAggctagctacaacgaATTGTTGGA-3′
td57
SEQ ID No: 127
5′-GCACAATCAggctagctacaacgaCTGGGTCAC-3′
td58
SEQ ID No: 128
5′-GGAGCACAAggctagctacaacgaCATCTGGGT-3′
td59
SEQ ID No: 129
5′-ACTGGAGCAggctagctacaacgaAATCATCTG-3′
td60
SEQ ID No: 130
5′-ATGGAGGGAggctagctacaacgaTGGAGCACA-3′
td61
SEQ ID No: 131
5′-TGGTACTTAggctagctacaacgaGGAGGGACT-3′
td62
SEQ ID No: 132
5′-GGGCTGGTAggctagctacaacgaTTATGGAGG-3′
td63
SEQ ID No: 133
5′-TCAACGATAggctagctacaacgaGCAGCCGGG-3′
td64
SEQ ID No: 134
5′-CCTCAACGAggctagctacaacgaATGCAGCCG-3′
td65
SEQ ID No: 135
5′-TCACCTCAAggctagctacaacgaGATATGCAG-3′
td66
SEQ ID No: 136
5′-CGTCGTTCAggctagctacaacgaCTCAACGAT-3′
td67
SEQ ID No: 137
5′-GTAAAGATAggctagctacaacgaGCGTGTTGG-3′
td68
SEQ ID No: 138
5′-AAGTAAAGAggctagctacaacgaATGCGTGTT-3′
td69
SEQ ID No: 139
5′-GGCAATGAAggctagctacaacgaTGGGTTTCT-3′
td70
SEQ ID No: 140
5′-TCACGGCAAggctagctacaacgaGAACTGGGT-3′
td71
SEQ ID No: 141
5′-AGGCAGTCAggctagctacaacgaGGCAATGAA-3′
td72
SEQ ID No: 142
5′-ATCTCGGCAggctagctacaacgaTCTGGTAGG-3′
td73
SEQ ID No: 143
5′-GCTGAGTAAggctagctacaacgaCTCGGCATT-3′
td74
SEQ ID No: 144
5′-TATTATCAAggctagctacaacgaTTTCAGCTG-3′
td75
SEQ ID No: 145
5′-GGGTTATTAggctagctacaacgaCAATTTTCA-3′
td76
SEQ ID No: 146
5′-AAGGGGTTAggctagctacaacgaTATCAATTT-3′
td77
SEQ ID No: 147
5′-CTCCCGGAAggctagctacaacgaCCTTTGGCA-3′
td78
SEQ ID No: 148
5′-GTACATGGAggctagctacaacgaTCAAAGTTC-3′

An oligonucleotide having a sequence SEQ ID NO: 40, SEQ ID NO: 139 or SEQ ID NO: 140 as the hydrophilic active ingredient is particularly preferred.

In addition, the analyzed formulations 1 to 3 and the submicron emulsion have the following composition (WO=composition of the W/O primary emulsion; WOW: composition of the multiple emulsion):

| | | | [% by |
|---|---|---|---|
| | | | weight] |
| Formulation 1 | | | |
| WOW_242 | WO: | Abil EM 90 | 3 |
| | | Medium-chain triglycerides | 17 |
| | | MgSO4 * 7H2O | 0.7 |
| | | H2O (preserved as indicated | 79.3 |
| | | below) | |
| | WOW: | WO | 80 |
| | | Poloxamer 407 | 0.8 |
| | | H2O (preserved as indicated | 19.2 |
| | | below) | |
| | Preservation: | Potassium sorbate | 0.14 |
| | | Citric acid | 0.07 |
| Formulation 2 | | | |
| WOW_167 | WO: | Sorbitan monooleates | 10 |
| | | Viscous paraffin | 39.5 |
| | | Lecithin | 0.5 |
| | | NaCl | 0.185 |
| | | H2O (preserved as indicated | 49.815 |
| | | below) | |
| | WOW: | WO | 40 |
| | | Steareth-20 | 1 |
| | | H2O (preserved as indicated | 59 |
| | | below) | |
| | Preservation: | Parabens | 0.1 |
| Formulation 3 | | | |
| WOW_146 | WO: | Sorbitan monooleates | 10 |
| | | Viscous paraffin | 39.5 |
| | | Lecithin | 0.5 |
| | | MgSO4 * 7H2O | 0.77 |
| | | H2O (preserved as indicated | 49.23 |
| | | below) | |
| | WOW: | WO | 40 |
| | | Steareth-20 | 1 |
| | | H2O (preserved as indicated | 59 |
| | | below) | |
| | Preservation: | Parabens | 0.1 |
| Formulation | | | |
| Submicron | | Coco-caprylate/caprate | 5 |
| emulsion | | Cetearyl isononanoate | 5 |
| | | Ethyl oleate | 5 |
| | | Oleth-3 | 0.414 |
| | | Oleth-10 | 5.586 |
| | | Glycerol 85% | 3 |
| | | MgSO4•7H2O | 0.3 |
| | | H2O (preserved as indicated | 75.7 |
| | | below) | |
| | Preservation: | Potassium sorbate | 0.14 |
| | | Citric acid | 0.07 |

The diagram in FIG. 1 clearly shows that the multiple emulsions comprising the DNAzyme in the internal water phase W₂ offer protection to up to 70% of the oligonucleotide from DNase degradation, depending on the composition (columns 2 to 4). When the DNAzyme is brought in contact with the DNases only as an aqueous solution, degradation is 100% (column 1).

Based on the results, it can be seen that protection only exists when the DNAzyme is located in the internal water phase W₂ of a multiple emulsion (entrapment of the active ingredient); when the DNAzyme is incorporated into the external water phase W₁, 100% degradation takes place, as in the case of the submicron emulsion (see column 5 and column 6).

Entrapment may thus only be achieved when the emulsion, as described in the general execution of the test, is produced in a two-step process. In this case, a W/O primary emulsion is produced from the components of the oil phase, to include the associated emulsifier, and the aqueous DNAzyme solution (W₂). The W/O primary emulsion thus obtained is then added in the second step, together with optionally present further adjuvants, to a second (DNAzyme-free) external water phase W₁, wherein the multiple water-in-oil-in-water emulsion forms.

### 5) Scale-Up Experiments

So as to evaluate the scale-up capability of a multiple emulsion according to the invention, various variants of the base emulsion WOW_{—}1 are produced using different scales:

| | | Drop | | Phase |
|---|---|---|---|---|
| Formulation | Scale: | size | Viscosity | stability |
| Basic formulation WOW_1 | 2.5 kg | +++ | ++ | +++ |
| composition of the water | | | | |
| phase W₁ and W₂, | | | | |
| supplemented with: | | | | |
| 0.14% potassium sorbate | | | | |
| 0.07% citric acid | | | | |
| Basic formulation WOW_1 | 12 kg | +++ | ++ | +++ |
| composition of the water | | | | |
| phase W₁ and W₂, | | | | |
| supplemented with: | | | | |
| 0.14% potassium sorbate | | | | |
| 0.07% citric acid | | | | |
| Basic formulation WOW_1 | 2.5 kg | ++ | ++ | +++ |
| supplemented with: | | | | |
| 10% butylene glycol | | | | |
| Basic formulation WOW_1 | 2.5 kg | ++ | ++ | +++ |
| composition of the water | | | | |
| phase W₁ and W₂, | | | | |
| supplemented with: | | | | |
| 0.14% potassium sorbate | | | | |
| 0.07% citric acid | | | | |
| 3% glycerin | | | | |
| Comparison formulation 1: | 2.5 kg | − | − | − |
| W₂: MgSO4, NaCl | | | | (phase |
| Oil phase: viscous paraffin, | | | | separation |
| lecithin, sorbitan | | | | and/or phase |
| monooleate | | | | inversion) |
| W₁: Steareth-20 | | | | |
| Preservative (in the water | | | | |
| phases W₁ and W₂): benzoic | | | | |
| acid | | | | |
| Comparison formulation 2: | 2.5 kg | + | ++ | ++ |
| W₂: MgSO4, NaCl | | | | |
| Oil phase: viscous paraffin, | | | | |
| lecithin, sorbitan | | | | |
| monooleate | | | | |
| W₁: Steareth-20, Sepineo | | | | |
| 600 (acrylamide/sodium | | | | |
| acryloyldimethyl taurate | | | | |
| copolymer/isohexadecane/ | | | | |
| Polysorbate 80) | | | | |
| Preservative (in the water | | | | |
| phases W₁ and W₂): benzoic | | | | |
| acid | | | | |

+++ very good
++ good
+ satisfactory
− poor

When the emulsions thus produced are analyzed with respect to the long-term stability thereof, one finds that no drop in stability can be observed in those emulsions that are based on the basic formulation WOW_{—}1, even with experiment durations of up to 6 months. The analyzed multiple emulsions based on the basic formulation thus all have very good long-term stability.

Comparison formulation 1 does not supply a stable multiple emulsion at all; comparison formulation 2 is only stable for a few weeks.

### 6) Addition of Various Preservatives to the Basic Formulation of an Emulsion According to the Invention

### 6.1) Influence on the Stability of the Emulsion

So as to evaluate the stability of an emulsion according to the invention when various preservatives are added, the basic formulation WOW_{—}1 described in item 2) is mixed with different preservatives. For this purpose, the respective preservative(s) is/are added to the two aqueous phases W₁ and W₂ already during the production of the emulsions. Thereafter, the drop size, viscosity and phase separation are analyzed.

The following additives may be used as preservatives (the percentage information listed below relates to percent by weight based on the amount of water): potassium sorbate, citric acid, butylene glycol, benzoic acid, propylene acid, sorbic acid and glycerin, specifically:
0.14% potassium sorbate in conjunction with 0.07% citric acid;
0.2% potassium sorbate in conjunction with 0.1% citric acid;
10% butylene glycol;
11% butylene glycol in conjunction with 3% glycerin;
0.05% benzoic acid;
20% propylene glycol;
0.1% sorbic acid in conjunction with 3% glycerin; and
0.12% sorbic acid in conjunction with 3% glycerin.

As the experiments demonstrate, various preservatives and preservative mixtures may be used, without negatively impacting the stability of the multiple emulsion. This demonstrates that an emulsion according to the invention can be protected against microbial contamination using various preservatives or preservative mixtures, without the stability of the emulsion being impaired.

Basic formulation WOW_{—}1 comprising various preservatives:

| Composition of the emulsion: | Drop size | Viscosity | Phase stability |
|---|---|---|---|
| Emulsion A: | +++ | +++ | +++ |
| basic formulation WOW_1 | | | |
| Emulsion B: | +++ | ++ | +++ |
| WOW_1 with | | | |
| 0.14% potassium sorbate | | | |
| 0.07% citric acid | | | |
| Emulsion C: | + | + | +++ |
| WOW_1 with | | | |
| 0.2% potassium sorbate | | | |
| 0.1% citric acid | | | |
| Emulsion D: | +++ | ++ | +++ |
| WOW_1 with | | | |
| 10% butylene glycol | | | |
| Emulsion E: | ++ | ++ | +++ |
| WOW_1 with | | | |
| 11% butylene glycol | | | |
| 3% glycerin | | | |
| Emulsion F: | +++ | + | +++ |
| WOW_1 with | | | |
| 0.05% benzoic acid | | | |
| Emulsion G: | ++ | ++ | +++ |
| WOW_1 with | | | |
| 20% propylene glycol | | | |
| Emulsion H: | ++ | ++ | +++ |
| WOW_1 with | | | |
| 0.1% sorbic acid | | | |
| 3% glycerin | | | |
| Emulsion I: | ++ | + | +++ |
| WOW_1 with | | | |
| 0.12% sorbic acid | | | |
| 3% glycerin | | | |

Evaluation key:
+++ very good
++ good
+ satisfactory
− poor

### 6.2) Microbiological Challenge Tests

For an evaluation of the preservative action, a microbiological challenge test can be carried out by way of example for the multiple emulsions B, C, H and I shown in item 7.1). The challenge test is a standard method that is carried out in accordance with Pharmacopoea Europaea (Eur. Ph.). Very good results are obtained for all emulsions. This also demonstrates that a multiple emulsion according to the invention can be effectively protected against microbial contamination by the addition of preservative.

### 7) Internal Structure of a Multiple Emulsion According to the Invention

The images shown in FIGS. 2 and 3 were recorded by way of coherent anti-Stokes Raman scattering spectroscopy (CARS). Detected were symmetric CH₂ stretching vibrations at 2845 cm⁻¹, so that the lipid distribution of the respective sample is shown here.

The analysis included both the basic formulation WOW_{—}1 described in item 2) of the exemplary embodiments and another multiple emulsion, which contrary to the emulsion according to the invention comprises sorbitan monooleates as the internal, lipophilic emulsifier and Steareth-20 as the external, hydrophilic emulsifier, and an oil phase made of paraffin and lecithin.

It is apparent from the CARS images that the multiple emulsion obtained with the basic formulation WOW_{—}1 has a considerably more homogeneous internal structure having many finely distributed internal water drops (FIG. 3) than the comparison emulsion comprising sorbitan monooleate as the internal emulsifier and Steareth-20 as the external emulsifier, and paraffin and lecithin as the oil phase (FIG. 2). This improved internal structure of the basic formulation on which the multiple emulsion according to the invention is based appears to be responsible for the extraordinary stability and protective function of the emulsions according to the invention. This effect can essentially be attributed to the lipophilic emulsifier cetyl PEG/PPG-10/1 dimethicone. Moreover, the advantageous homogeneous structure appears to be responsible for the fact that, by way of the emulsion according to the invention, large amounts of an active ingredient, in particular large amounts of a DNAzyme, can be entrapped in the internal water phase W₂ of the emulsion.

### 8) DNAzyme Recovery

Based on the composition of the basic formulation WOW_{—}01 described in item 2) of the exemplary embodiments, the two exemplary embodiments V1 and V2, shown below, of an emulsion according to the invention can be produced by way of example. When the amount of DNAzyme present in the emulsions after production is analyzed by way of HPLC, the following results are obtained:

Composition of the analyzed emulsion V1:

Composition of the basic formulation WOW_{—}01, as well as additionally
in the internal water phase W₂: 0.4% of a DNAzyme (Na salt), potassium sorbate*/citric acid** as preservatives, and
in the external water phase W₁: potassium sorbate*/citric acid** as preservatives.
   * amount of potassium sorbate: 14% by weight based on the amount of water
   ** amount of citric acid: 0.07% by weight based on the amount of water

A recovery rate for the active ingredient (DNAzyme) (amount of active ingredient quantified by way of HPLC) of 96.67+/−2.89% was found.

Composition of the analyzed emulsion V2:

Composition of the basic formulation WOW_{—}01, as well as additionally
in the internal water phase W₂: 0.4% of a DNAzyme (Na salt), potassium sorbate*/citric acid**/glycerin*** as preservatives, and
in the external water phase W₁: potassium sorbate*/citric acid**/glycerin*** as preservatives.
   * amount of potassium sorbate: 14% by weight based on the amount of water
   ** amount of citric acid: 0.07% by weight based on the amount of water
   *** amount of glycerin: 3% by weight based on the amount of water

A recovery rate for the active ingredient (DNAzyme) (amount of active ingredient quantified by way of HPLC) of 102.00 +/−6.10% was found.

The invention is not limited to one of the embodiments described above, but may be modified in a variety of ways.

Depending on the usage purpose, for example, it is conceivable for the multiple emulsion according to the invention to comprise additional constituents, which are provided to improve the skin care properties, consistency, viscosity and/or penetration of the active ingredient.

These include—unless they are already provided as the active ingredient—for example, humectants, conditioning agents and mineral nutrients such as glycerin, urea, allantoin, inositol, sugar, amino acids, sodium lactate, water-soluble magnesium, sodium and/or potassium salts, which are preferably present in a content of 0.1 to 8% by weight, and particularly preferably in a content of 3% by weight, based on the multiple emulsion, vitamins such as retinol, thiamine, ascorbic acid, calciferol, riboflavin, tocopherol, cobalamin, panthothenic acid, biotin, pyridoxin, niacin and/or folic acid, which are preferably present in a content of 0.01 to 5% by weight, based on the multiple emulsion, nourishing natural oils such as evening primrose and/or avocado oil, as well as paraffin oil for occlusion either alone or in a mixture with medium-chain triglycerides, thickeners such as carbomers, cellulose derivatives, xanthan, polymers and/or polyethylene glycols.

Substances that increase penetration are preferably used in the oil phase. These include: lecithin, which is preferably present in a content of 0.1 to 3% by weight based on the multiple emulsion, and/or fatty acid esters of oleic acid, preferably decyl oleate or oleyl oleate, in which preferably a content of 0.1 to 5% by weight based on the multiple emulsion is present, one or more monohydric or polyhydric alcohols, in particular propylene glycol, which are preferably present in a content of 0.1 to 20% based on the multiple emulsion.

In addition to the active ingredient that is provided according to the invention, the multiple emulsion may comprise additional active ingredients. These do not necessarily all have to be present in the internal water phase W₂, but may also be included in the oil phase O or the external water phase W₁. In addition to the hydrophilic active ingredient that is provided according to the invention, it is thus also possible for multiple lipophilic active ingredients and/or further hydrophilic active ingredients to be present.

All of the features and advantages that are apparent from the claims, the description and the drawings, including design details, arrangements in terms of space, and method steps, can be essential to the invention, both alone and in a wide variety of combinations.

1. A multiple emulsion for applying at least one pharmaceutical or cosmetic active ingredient, comprising an external water phase W₁, an oil phase O dispersed in the external water phase W₁ and an internal water phase W₂ dispersed in the oil phase O, at least one electrolyte selected from the group of alkali metal and alkaline earth metal halides and sulfates and at least one hydrophilic active ingredient being provided in the internal water phase W₂, characterized in that
the external water phase W₁ comprises a hydrophilic emulsifier, which is a polymer of ethylene oxide and propylene oxide, and
the oil phase O is formed by triacylglycerides and comprises a lipophilic emulsifier from the group of dimethicones.

2. The multiple emulsion according to claim 1, characterized in that the hydrophilic emulsifier is a poloxamer.

3. A multiple emulsion according to claim 1, characterized in that the lipophilic emulsifier is a linear dimethicone having polyether and alkyl groups.

4. A multiple emulsion according to claim 1, characterized in that the triacylglycerides are esters of saturated fatty acids.

5. A multiple emulsion according to claim 1, characterized in that the electrolyte is NaCl, MgSO₄ or a mixture of NaCl and MgSO₄.

6. A multiple emulsion according to claim 1, characterized in that the hydrophilic active ingredient is MgSO₄.

7. A multiple emulsion according to claim 1, characterized in that the hydrophilic active ingredient is an oligonucleotide, preferably an oligonucleotide having a sequence according to one of the sequences SEQ ID NO: 1 to SEQ ID NO: 148 or SEQ ID NO: 150.

8. A multiple emulsion according to claim 1, characterized in that the multiple emulsion comprises one or more preservatives.

9. A multiple emulsion according to claim 1, characterized in that the drop size of the dispersed oil phase is 1 μm to 100 μm.

10. A multiple emulsion according to claim 1, characterized in that a saturated hydrocarbon is present in the oil phase.

11. A multiple emulsion according to claim 1, characterized in that the hydrophilic emulsifier is present in the external water phase W₁ in a content of 0.1 to 5% by weight.

12. A multiple emulsion according to claim 1, characterized in that the lipophilic emulsifier, based on a W/O primary emulsion consisting of the oil phase O and the internal water phase W₂, is present in a content of 0.1 to 6% by weight.

13. A multiple emulsion according to claim 1, characterized in that the triglycerides, based on a W/O primary emulsion consisting of the oil phase O and the internal water phase W₂, are present in a content of 5 to 30% by weight.

14. A multiple emulsion according to claim 1, characterized in that the electrolyte, based on a W/O primary emulsion consisting of the oil phase O and the internal water phase W₂, is present in a content of 0.1 to 3% by weight.

15. A multiple emulsion according to claim 1, characterized in that the hydrophilic active ingredient is present in a content of 0.01 to 10% by weight, based on the total mass of the multiple emulsion.

16. A multiple emulsion according to claim 1, characterized in that
the hydrophilic emulsifier in the external water phase W₁ has a content of 0.1 to 5% by weight; and
the lipophilic emulsifier, based on a W/O primary emulsion consisting of the oil phase O and the internal water phase W₂, is present in a content of 0.1 to 6% by weight; and
the triglycerides, based on the W/O primary emulsion, are present in a content of 5 to 30% by weight; and
the electrolyte, based on a W/O primary emulsion consisting of the oil phase O and the internal water phase W₂, is present in a content of 0.1 to 3% by weight; and
the hydrophilic active ingredient, based on the total mass of the multiple emulsion, is present in a content of 0.01 to 10% by weight; and
a preservative is present in the external water phase W₁ and/or in the internal water phase W₂ in a content of 0 to 20% by weight.

17. Use of a multiple emulsion according to claim 1 for producing a pharmaceutical, a medicinal product or a cosmetic.

18. Use according to claim 17, characterized in that the multiple emulsion is used as a drug vehicle.

19. A pharmaceutical, comprising a multiple emulsion according to claim 1 for topical use, or for application of the active ingredient by way of mucous membranes, or for intraperitoneal application of the active ingredient.

20. The pharmaceutical according to claim 18, characterized in that the multiple emulsion is a drug vehicle.

21. A pharmaceutical comprising a multiple emulsion according to claim 1 for treating an inflammatory disease
